(19) United States
(12) Patent Application Publication
(10) Pub. No.: US 2003/0050616 A1
(43) Pub. Date: Mar. 13, 2003

(54) ABSORBENT ARTICLE HAVING A DEPLOYABLE GASKET ELEMENT

(76) Inventors: GARY M. REYNOLDS, WOODSTOCK, GA (US); PATRICK MCNICHOLS, HORTONVILLE, WI (US)

Correspondence Address: PAULEY PETERSEN KINNE & FEJER, 2800 WEST HIGGINS ROAD, SUITE 365, HOFFMAN ESTATES, IL, 60195

(21) Appl. No.: 09/399,630

(22) Filed: Sep. 20, 1999

(51) Int. Cl. A61F 13/15

(52) U.S. Cl. 604/369

(57) ABSTRACT

An absorbent article adapted to fit about the waist of a wearer and having front and rear waist sections, an intermediate section and a gasketing assembly operatively joined with a first portion of the rear waist section. A first portion of the rear waist section is formed of a stretchable material to which the gasketing assembly is operatively joined. Upon tensioning of the stretchable material, the gasketing assembly deploys a gasket element configured to inhibit a longitudinal flow of human discharge along a bodyfacing surface of the absorbent article.

### FIELD OF THE INVENTION

This invention relates generally to absorbent articles for absorbing body fluids and exudates, such as urine and fecal material, for example. More particularly, the present invention relates to absorbent garments, such as disposable diapers, training pants for children and adult incontinence garments, which are configured to collect and contain fecal material and desirably avoid leakage.

### BACKGROUND OF THE INVENTION

Conventional absorbent articles, such as disposable diapers, employ absorbent materials located between a liquid pervious topsheet and a liquid impermeable backsheet to absorb body exudates. Such conventional absorbent articles have also typically included elasticized waistbands and leg cuffs to help reduce the leakage of body exudates.

Conventional absorbent articles, however, have not always been completely satisfactory. For example, many diaper form conventional absorbent articles have not completely contained body exudates within the article during use thereby undesirably resulting in leakage such as may soil the clothes of the wearer. Such leakage problems have been particularly evident in the waist margins of the absorbent articles when runny or watery fecal material has been excreted by the wearer during use. Typically, such runny or watery fecal material has been forced longitudinally outwards from the crotch of the article due to forces exerted by the wearer. Such leakage problems are magnified when the wearer is particularly active and may thus exert forces of relatively large magnitude on the crotch area of the article. Such leakage problems may also, at least in part, occur as a result of the tendency of such excreted fecal material to travel or otherwise be conveyed along or adjacent the skin of the wearer and thus not be sufficiently or adequately contained and controlled within a particular absorbent article.

Some conventional absorbent articles have included elasticized components and containment or barrier flaps at the waist sections of the article to reduce the occurrence of such leakages. However, such elasticized components and containment flaps generally have not completely eliminated all leakage from the waist sections of such associated articles. For example, exudates such as runny fecal material may remain on the skin of the wearer and be transferred therealong until such time the exudate material may escape through small openings between the containment flaps and the body of the wearer. Such openings between the body of the wearer and the containment flaps may be created or caused due to either or both improper fit of the absorbent article about the wearer and the movements of the wearer during use. Also, perimeter tension of such absorbent articles may undesirably create open voids in areas where the body of the wearer assumes a concave form, e.g., the area spanning the separation between the small of the back and the gluteal fold.

As a result, although the inclusion of waist elastics and containment flaps have generally improved the performance of such absorbent articles, there remains a need to further reduce either or both the number and magnitude of leaks and, in particular, either or both the number and magnitude of leaks of fecal material from the waist sections of such absorbent articles.

To that end, U.S. Pat. No. 5,833,677, the disclosure of which is hereby incorporated by reference in its entirety, specifically discloses an absorbent article which includes a compression resistant containment dam configured to inhibit the longitudinal flow of fecal material along the bodyfacing surface of the absorbent article. As disclosed, the containment dam is generally positioned in a laterally extending direction and is located on the bodyfacing surface of the absorbent article such that, when the absorbent article is being worn by a wearer sitting on a flat surface, the containment dam is positioned along a line where the wearer's buttocks depart from the flat surface.

While such an absorbent article represents an improvement over earlier forms of absorbent articles and can be generally effective for the intended purpose of reducing, minimizing or avoiding leakage therefrom of at least certain body exudates, further improvements may be desired or required at least for particular absorbent article uses.

Thus, there is a need and a demand for absorbent articles of improved efficiency in reducing, minimizing or avoiding leakage therefrom of at least certain body exudates.

### SUMMARY OF THE INVENTION

A general object of the invention is to provide an improved absorbent article and associated method.

A more specific objective of the invention is to overcome one or more of the problems described above.

The general object of the invention can be attained, at least in part, through an absorbent article, such as adapted to fit about the waist of a wearer and having a longitudinal direction and a lateral direction. In accordance with one preferred embodiment of the invention, such an absorbent article includes front and rear waist sections, an interconnecting intermediate section and a gasketing assembly. At least a first portion of the rear waist section is formed of a stretchable material. The intermediate section interconnects the front and rear waist sections and includes an absorbent portion. The gasketing assembly is operatively joined with the first portion of the rear waist section to mechanically deploy, upon tensioning of the stretchable material, a gasket element configured to inhibit a longitudinal flow of human discharge along a bodyfacing surface of the absorbent article.

The prior art fails to provide absorbent articles, particularly in the nature of disposable absorbent articles such as diapers, for example, which are as effective as may be desired in preventing or avoiding the passage of human discharge along the bodyfacing surface of the absorbent article.

The invention further comprehends an improved disposable absorbent article. Such a disposable article defines a longitudinal direction and a lateral direction and includes a stretchable waist material adapted to fit about the waist of a wearer. In accordance with one embodiment of the invention, such an improved disposable article includes a gasketing assembly operatively joined with the stretchable waist material to deploy a gasket element against the lower back of a wearer when the waist material is in a stretched conditioned. The deployed gasket element is generally effective to inhibit flow of matter between the waist of the wearer and the waist material of the absorbent article.

The invention still further comprehends a method for use in association with an absorbent article having a longitudinal direction and a lateral direction and which absorbent article includes a front waist section, a stretchable rear waist section, and an intermediate section which interconnects the front and rear waist sections and which intermediate section includes an absorbent portion. In accordance with one embodiment of the invention, such method includes tensioning the stretchable rear waist section to deploy a gasket element for inhibiting a longitudinal flow of human discharge along a bodyfacing surface of the absorbent article.

Other objects and advantages will be apparent to those skilled in the art from the following detailed description taken in conjunction with the appended claims and drawings.

### BRIEF DESCRIPTION OF THE DRAWINGS

FIG. 1 a simplified perspective view of an absorbent article which includes a gasketing assembly in accordance with one embodiment of the invention.

FIG. 2 is a detailed fragmentary view taken substantially along the line 2-2 of FIG. 1 and viewed in the direction of the arrows.

FIG. 3 is a simplified fragmentary schematic view of the absorbent article shown in FIGS. 1 and 2, taken substantially along the line 3-3 of FIG. 1 and viewed in the direction of the arrows, showing the gasket assembly joined with a waist section portion of the absorbent article in a relaxed condition or state.

FIG. 4 is a simplified fragmentary schematic view, similar to that shown in FIG. 3, but now with the waist section portion of the absorbent article in a tensioned condition or state.

FIG. 5 is a simplified fragmentary schematic view, similar to that shown in FIG. 4, showing a waist section portion of an absorbent article in accordance with another embodiment of the invention in a tensioned condition or state.

FIG. 6 is a simplified fragmentary schematic view, similar to that shown in FIG. 4, showing a waist section portion of an absorbent article in accordance with yet another embodiment of the invention in a tensioned condition or state.

FIG. 7 is a simplified fragmentary schematic view, similar to that shown in FIG. 4, showing a waist section portion of an absorbent article in accordance with yet still another embodiment of the invention in a tensioned condition or state.

FIG. 8 is a simplified fragmentary schematic view, similar to that shown in FIG. 4, showing a waist section portion of an absorbent article in accordance with an alternative embodiment of the invention in a tensioned condition or state.

FIG. 9 is a simplified fragmentary schematic view, similar to FIG. 3, showing a waist section portion of an absorbent article in a relaxed condition or state, in accordance with another alternative embodiment of the invention.

FIG. 10 is a simplified fragmentary view showing the waist section portion of the absorbent article of FIG. 9 but now in a tensioned condition or state.

FIG. 11 is a simplified fragmentary schematic view, similar to that shown in FIG. 4, showing a waist section portion of an absorbent article in accordance with yet another alternative embodiment of the invention in a tensioned condition or state.

FIG. 12 is a simplified fragmentary schematic view, similar to FIG. 3, showing a gasket assembly joined with a waist section portion of an absorbent article in a relaxed condition or state, in accordance with another alternative embodiment of the invention.

FIG. 13 is a simplified fragmentary schematic view showing the gasket assembly joined with the waist section portion of the absorbent article, as shown in FIG. 12, but now with the waist section portion of the absorbent article in a tensioned condition or state.

### DETAILED DESCRIPTION OF THE INVENTION

The present invention, as is described in more detail below, provides an improved absorbent article and associated method such as may desirably inhibit the longitudinal flow along a bodyfacing surface thereof.

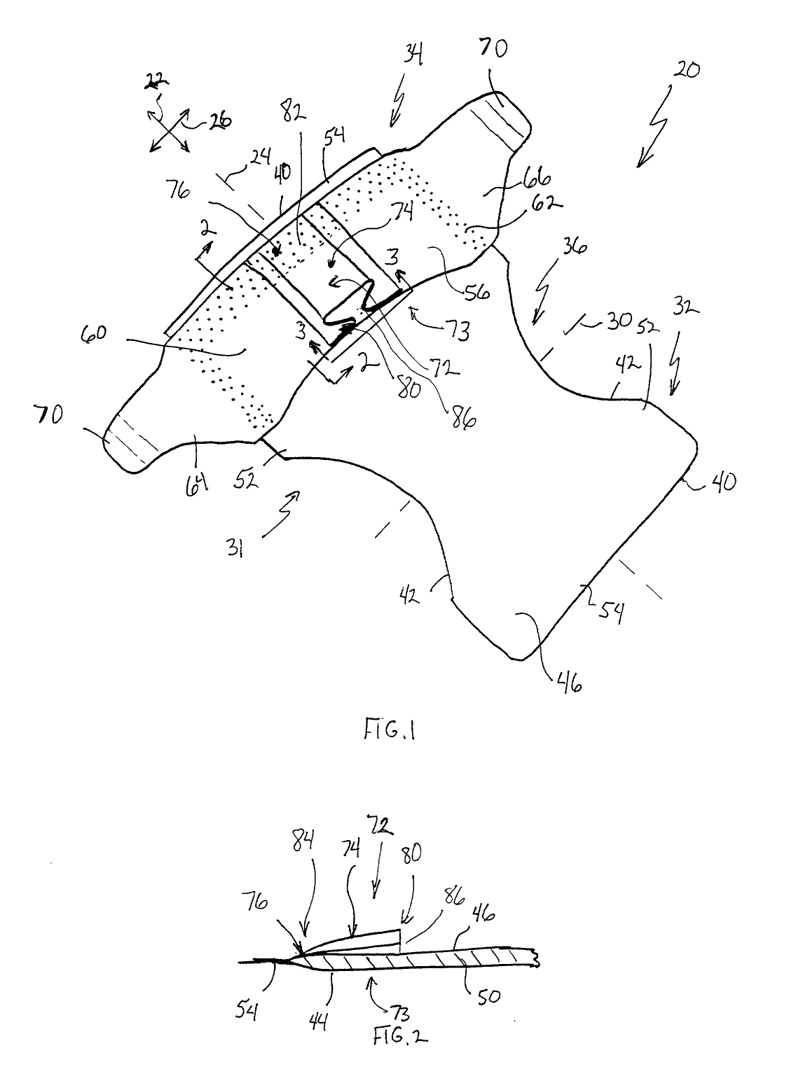

Referring initially to FIGS. 1-4, there is illustrated an absorbent article, generally designated by the reference numeral 20, in accordance with one embodiment of the invention. As will be appreciated, the absorbent article 20 has the general form of a disposable diaper such as adapted to be worn by infants about the lower torso. It is to be understood, however, that while the invention is described below with particular reference to disposable diapers, the broader practice of the invention is not necessarily so limited. For example, the invention can, if desired, be applied to other forms or types of absorbent articles including various disposable absorbent articles such as are generally configured to collect and contain human discharges or exudates such as, including, urine and fecal material and which articles also desirably avoid leakage of such discharge materials. In particular, specific absorbent articles in accordance with the invention can find application in or as feminine care pads, incontinence garments, training pants, and the like, for example. In addition, the invention will be described below in the context of various configurations. It is to be appreciated that alternative arrangements of the invention can comprise any combination of such configurations.

The diaper 20 may be of various suitable shapes. For example, the diaper may have an overall rectangular shape, T-shape or an approximately hourglass shape. In the illustrated embodiment, the diaper absorbent article 20 has a longitudinal direction 22, with a longitudinal centerline 24 and a lateral direction 26 with a lateral centerline 30. The absorbent article 20 includes a body or chassis 31. The absorbent article 20 also generally defines a front waist section 32, a rear waist section 34, an intermediate section 36 which interconnects the front and rear waist sections, a pair of longitudinally opposed end edges 40 and a pair of opposed lateral side edges 42. The front and rear waist sections 32 and 34, respectively, include the general portions of the article which are constructed to extend substantially over the front and rear abdominal regions, respectively, of the wearer during use. The intermediate section 36 of the article includes the general portion of the article which is constructed to extend over and about the crotch region, between the legs, of the wearer. The opposed end edges 40 define a waist opening for the diaper 20 and typically are straight but may also be curvilinear. The opposed side edges 42 define leg openings for the diaper and generally are typically curvilinear or contoured to more closely fit the legs of the wearer.

The diaper 20 includes a substantially liquid impermeable backsheet 44, a porous, liquid permeable topsheet 46 positioned in facing relation with the backsheet 44, and an absorbent body 50, such as an absorbent pad, which is located between the backsheet 44 and the topsheet 46. Such various components of the diaper chassis 31 are individually and collectively well known to those skilled in the art. Further, such components may be integrally assembled together employing various types of suitable attachment means, such as adhesive, sonic bonds, thermal bonds or combinations thereof. For example, as shown, the backsheet 44 and the topsheet 46 may be assembled to each other and to the absorbent body 50 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Other methods or techniques for combining or joining the various components will be apparent to those skilled in the art and may be herein employed.

If desired, marginal portions of the diaper 20, such as marginal sections of the backsheet 44 and the topsheet 46, may extend past the terminal edges of the absorbent body 50. For example, the backsheet 44 and the topsheet 46 may extend outwardly beyond the terminal marginal edges of the absorbent body 50 such as to form side margins 52 and end margins 54, respectively. The topsheet 46 is generally coextensive with the backsheet 44 but may optionally cover an area which is larger or smaller than the area of the backsheet 44, as desired.

As will be appreciated, diapers in accordance with the invention may, if desired, include features such as elasticized members (not shown) such as leg elastic members and waist elastic members or the like, such as known in the art to facilitate or improve proper fit and/or to help reduce leakage of body exudates from the diaper. For example, leg elastic members may be included as single or multiple strands of elastic or elastomeric composites which are constructed to operably gather and shirr the diaper 20 to provide elasticized leg bands which can closely fit around the legs of the wearer to reduce leakage and provide improved comfort and appearance. Similarly, waist elastic members can be employed to elasticize the ends of the diaper to provide elasticized waistbands such as configured to operably gather and shirr the waistband sections to provide a resilient, comfortably close fit around the waist of the wearer.

The rear waist section 34 of the absorbent article 20 includes a stretchable material 56. In the illustrated embodiment, the stretchable material 56 is in the form of a band 60. As will be appreciated, various stretchable materials are useable in the practice of the invention including woven or nonwoven elastic or gathered extensible materials such as copolymers of polystyrene, polyisoprene or polybutadiene, copolymers of ethylene, natural rubbers, urethanes, and coextrusions/blends of the aforementioned, for example. Extensible materials useable in the practice of the invention need not have retractable properties. Further, the above materials and the like may be formed by any suitable process, such as film extrusion, spunbond process, meltblow process, laminate process or the like.

The stretchable band 60 is joined or bonded with the chassis 31, such as via the application of ultrasonic energy as is known in the art, and such as represented by the ultrasonic bonds 62. It is to be understood, however, that the broader practice of the invention is not limited by or to the specific form or means by which such or similar stretchable material is included or incorporated into a particular absorbent article. In particular, other means or forms for the inclusion of such stretchable materials or the like will be apparent to those skilled in the art and guided by the teachings herein provided and such other means or forms are also herein encompassed.

The stretchable band 60 has formed or joined at opposed first and second ends thereof, 64 and 66, respectively, a pair of fasteners 70 which are employable to secure the diaper 20 about the waist of the wearer. Suitable fasteners 70 may include hook-and-loop type fasteners, adhesive tape fasteners, buttons, pins, snaps, mushroom-and-loop fasteners and the like as may be desired for particular applications.

In accordance with the invention, a gasketing assembly, generally designated by the reference numeral 72, is operatively joined with the stretchable band 60 at or about a rear waist portion 73 of the absorbent article 20. The gasketing assembly 72 includes a specifically folded or shaped gasket element 74, preferably positioned or centered at or about the longitudinal centerline 24 and such as formed of a generally nonstretchable material such as a nonwoven fabric, such as nonwoven SMS (spunbond-meltblown-spunbond) laminate material; a meltblown fabric such as composed of meltblown fibers, such as meltblown polypropylene fibers; a woven fabric or a polymer film or the like.

As shown in FIGS. 1 and 2, the gasket element 74 has opposed first and second longitudinal edges, 76 and 80, respectively. As shown, such gasket element first longitudinal edge 76 is generally at or adjacent the rear end edge 40 and is bonded or otherwise joined at the rear waist section 34 such as via ultrasonic bonding, as represented by the ultrasonic bonds 82 or the like to form a substantially closed rear waist end portion 84. The gasket element second longitudinal edge 80, as described in greater detail below, is desirably at least initially maintained unattached or open, such as to form an open crotch-extending end 86.

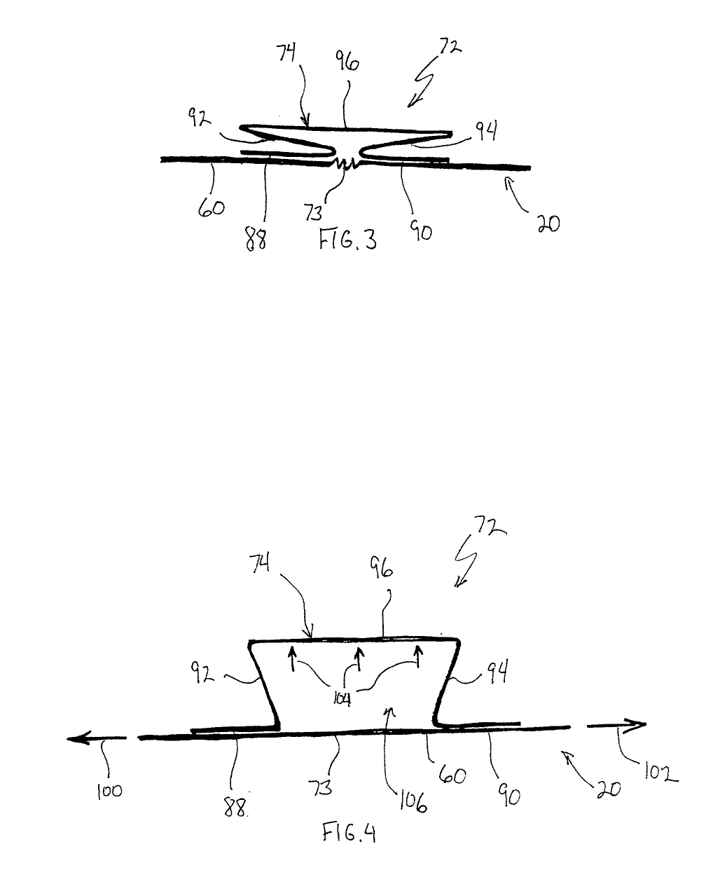

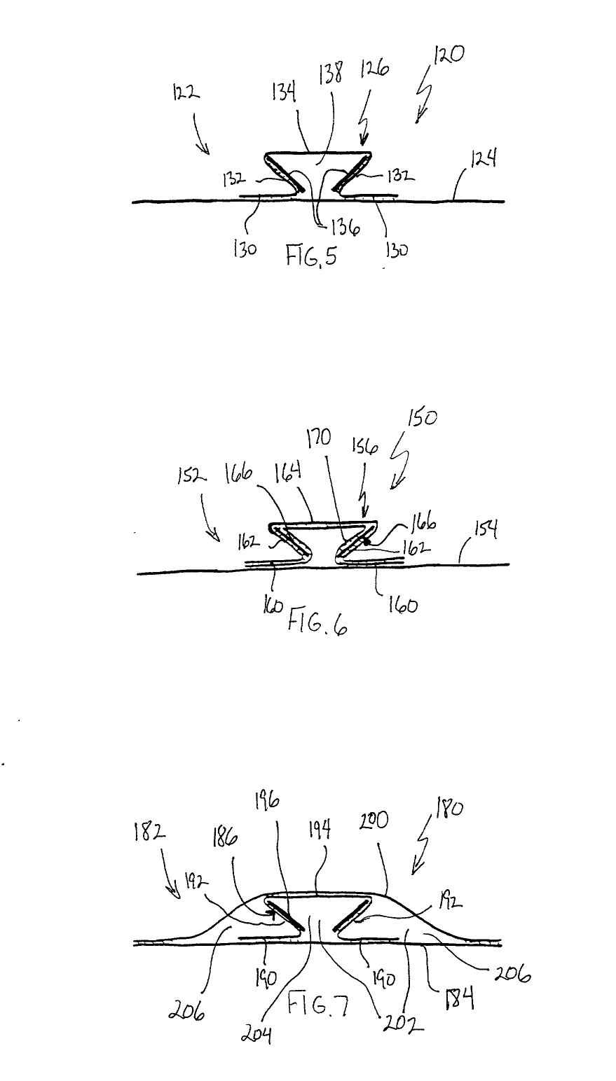

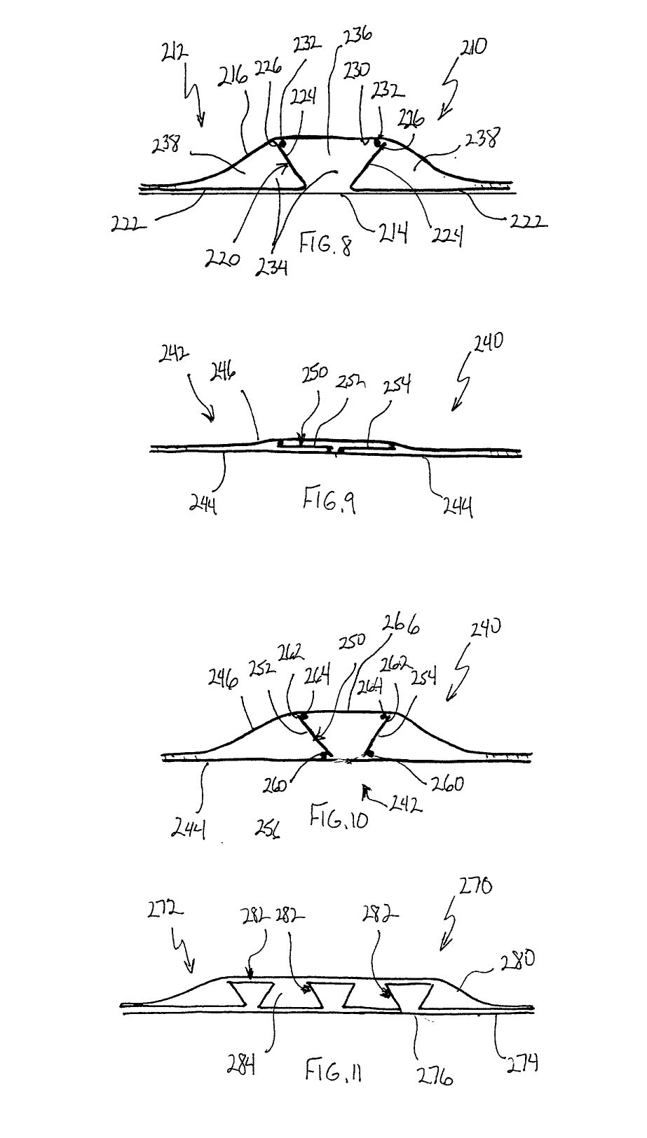

Turning now to FIGS. 3 and 4, the gasketing assembly 72 and the operation thereof will be more specifically described. In particular, FIG. 3 generally illustrates the gasketing assembly 72 joined with the rear waist section portion 73 of the absorbent article 20, specifically, the stretchable band 60, in a relaxed condition or state. FIG. 4 generally illustrates the gasketing assembly 72 joined with the rear waist section portion 73 of the absorbent article 20, specifically, the stretchable band 60, in a tensioned condition or state.

The gasket assembly element or member 74 includes a pair of opposed base portions 88 and 90, respectively, a pair of leg or thrust portions 92 and 94, respectively, and at least one face portion 96. While the gasket element 74 may, as illustrated, be formed continuously of a single material such that the base portions 88 and 90, the leg portions 92 and 94 and the face portion 96 are formed in one continuous piece the broader practice of the invention is not necessarily so limited. For example, a member can, if desired, be formed of two or more pieces such as of different of the same material and such as may be attached or otherwise joined together, such as to provide specific constructions and performance capabilities for particular applications as will be apparent to those skilled in the art and guided by the teachings herein provided.

The base portions 88 and 90 are operativelyjoined or attached to the rear waist section 34, specifically to the rear waist band 60 such as by means of an adhesive or other suitable form of joinder. Further, the base portions 88 and 90 generally lie adjacent and parallel to the band 60 both in the relaxed condition or state, shown in FIG. 3, and in the tensioned condition or state, shown in FIG. 4. Further, in the relaxed condition or state shown in FIG. 3, at least a portion of the stretchable band 60, specifically at least a portion of the stretchable band between the gasketing assembly member base portions 88 and 90 is in a contracted or relaxed state. Further in the relaxed condition or state shown in FIG. 3, the leg portions 92 and 94 and the face portion 96, respectively, generally lie flat and respectively adjacent to the base portions 88 and 90 such as in an accordion fold fashion. However, tensioning of the stretchable band 60, as signified by the arrows 100 and 102, shown in FIG. 4, generally results in the leg portions 92 and 94 assuming a displacement having at least a component perpendicular to the base portions 88 and 90, respectively, and thus the rear waist section 73.

As will be appreciated, with the leg portions 92 and 94 so more generally perpendicularly disposed relative to the base portions 88 and 90, the face portion 96 of the gasketing assembly element 74 is thrust or otherwise projected, as signified by the arrows 104, in a direction away from the stretchable band 60 and towards, preferably adjacent, the body of the wearer. Such deployment of the gasketing assembly 72 desirably serves to form or create a containment volume, designated by the reference numeral 106 (shown in FIG. 4) adjacent the open crotch-extending end 86, shown in FIGS. 1 and 2 and effective to contain or hold body exudates therewithin and spaced apart from contact with the body of the wearer.

Desirably, the containment volumes of the different aspects of the present invention are configured to contain substantially all of the solid fecal exudates in the target zone of the diaper 20. As used herein, the term “target zone” refers to that portion of the diaper 20 which is configured to directly receive the insult of fecal exudates from the wearer and generally is along the longitudinal centerline 24, shown in FIG.1.

Typically, the gasketing assembly 72 is located in the rear waist section 34 of the diaper 20 and extends in the lateral direction 26 and in the longitudinal direction 24 towards and possibly into the intermediate section 36 of the diaper 20.

In practice, such deployment of the gasket element 74 adjacent the body of the wearer such as to occupy or otherwise fill the volume normally occurring between the waist band of the absorbent article and the small of the back of the wearer serves to reduce the potential for leakage from the absorbent article at such location. Thus, the invention may desirably provide an arrangement which utilizes perimeter tension, such as associated with either or both the placement and securing of an absorbent article about the waist of a wearer, to actuate or effect the desirable deployment and positioning of the gasket assembly element 74 into desired or proper position such as may be effective to reduce, minimize or avoid leakage from the associated absorbent article at the particular location thereof.

Thus, absorbent articles in accordance with the invention are generally configured to remove exudates such as runny fecal material from the skin of the wearer and inhibit the longitudinal flow of fecal exudates into regions of the diaper 20, such as at the rear waist section 34, to more effectively contain such material within the diaper 20.

FIGS. 5-13 schematically illustrate certain currently contemplated alternative preferred embodiments for effecting desired gasketing assembly member deployment, in accordance with the invention.

More specifically, FIG. 5 illustrates an absorbent article assembly, generally designated by the reference numeral 120. The absorbent article assembly 120 includes a rear waist section 122 formed, at least in part, of a stretchable material band 124, such as described above such as composed of an elastomeric or gathered material, for example, and having a gasketing assembly element 126 joined thereto. The gasketing assembly element 126 includes base portions 130, leg portions 132 and a face portion 134, such as described above.

In the gasketing assembly element 126, at least the leg portions 132 include a rigid or otherwise effective compression resistant member 136 such as composed of a plastic sheet material such as polyurethane, polyethylene or the like or a stiff paper product such as cardboard or the like. Thus, upon tensioning of the stretchable band 124, such leg portions 132 are effective to mechanically convert such perimeter tension to deploy the face portion 134 towards, preferably adjacent, the body of the wearer and such as may form or create a volume, designated by the reference numeral 138, effective to contain or hold body exudates therewithin and spaced apart from contact with the body of the wearer.

FIG. 6 illustrates an absorbent article assembly 150 in accordance with another specific embodiment of the invention. The absorbent article assembly 150 is generally similar to the absorbent article assembly 120 described above in that it includes a rear waist section 152 formed, at least in part, of a stretchable material band 154, such as described above, with a gasket assembly member 156 joined thereto. The gasket member 156 includes base portions 160, leg portions 162 and a face portion 164 with the leg portions 162 including a rigid or otherwise compression resistant member 166.

The absorbent article assembly 160 differs from the absorbent article assembly 120, however, in that the compression resistant member 166 is encased, as a part of the gasket assembly member 156, by an inner covering 170 such as formed of soft nonwoven material such as may result in a more wearer friendly form.

While the above-described embodiments utilize gasketing assemblies wherein the gasket member or element is bonded, joined or otherwise attached at the rear waist section 34 to form a substantially closed rear waist end portion 84, as shown in FIG. 1, it is to be understood that the broader practice of the invention is not necessarily so limited. For example, FIG. 7 illustrates an absorbent article assembly 180, in accordance with one such alternative embodiment of the invention. More specifically, the absorbent article assembly 180 in addition to a rear waist section 182 formed, at least in part, of a stretchable material band 184, also includes a gasket assembly member 186 joined thereto.

The gasket assembly member 186, similar to those described above, includes base portions 190, leg portions 192 and a face portion 194, with the leg portions 192 including a compression resistant member 196. The absorbent article assembly 180, however, additionally includes a bodyside liner 200, such as formed by a soft spunbond material overlying the gasketing assembly member 186. As will be appreciated, in the normal use of an absorbent article which includes such a bodyside liner 200, the bodyside liner is normally placed adjacent the body of the wearer. Further, the bodyside liner 200 may have at least a segment of the rear waist end thereof (not shown) bonded, joined or otherwise attached at the rear waist section of the absorbent article to form a substantially closed rear waist end portion.

With the absorbent article assembly 180, tensioning of the stretchable band 184 serves to effect deployment of the gasket assembly face portion 194 towards the body of the wearer and thus, in turn, projection of the bodyside liner 200 towards, preferably adjacent, the body of the wearer and such as may form or create a volume, designated by the reference numeral 202, effective to contain or hold body exudates therewithin and spaced apart from contact with the body of the wearer. As shown, such containment volume 202 includes a first volume portion 204 within the deployed gasket assembly member 186, e.g., spanning between the leg portion 192, and a second volume portion 206 within the bodyside liner 200 and external the deployed gasketing assembly member 186. Thus, in at least in certain preferred embodiments, the respective gasket assembly element need not be joined to the absorbent article along the entire lateral length thereof.

Various alternative embodiments of assemblies in accordance with the invention are contemplated and herein encompassed including, but not necessarily limited to those shown in FIGS. 8-13 and described in further detail hereinafter.

For example, FIG. 8 illustrates an absorbent article assembly 210 which includes a rear waist section 212 formed, at least in part, by a stretchable material band 214. The absorbent article assembly 210 additionally includes a bodyside liner 216, such as described above, with a gasketing assembly 220, in accordance with a specific alternative preferred embodiment of the invention disposed therebetween.

More specifically, the gasketing assembly 220 includes base portions 222, such as bonded or otherwise joined or connected to the stretchable material band 214. The gasketing assembly 220 in addition includes leg portions 224 which, rather than being joined or connected together via a gasketing assembly face portion, each include a terminal end 226 which is joined or secured to a face 230 of the bodyside liner 216, e.g., the face of the bodyside liner 216 generally opposite the wearer when the absorbent article is normally worn. In the illustrated embodiment, such joinder is effected by means of an adhesive bead 232 but, as will be appreciated by those skilled in the art, other suitable means of joinder or attachment can be sued and are herein encompassed.

In the absorbent article assembly 210, tensioning of the stretchable material band 214 generally results in the leg portions 224 assuming a displacement having at least a component perpendicular to the stretchable waist material 214 and the bodyside liner 216 and thus desirably serve to push or deploy of the bodyside liner 216 towards, preferably adjacent, the body of the wearer and such as may form or create a containment volume 234 effective to contain or hold body exudates, e.g., fecal material, therewithin and spaced apart from contact with the body of the wearer. As shown, such containment volume 234 includes a first volume portion 236 spanning between the deployed leg portion 224, and a second volume portion 238 within the bodyside liner 216 and external the base portions 222. Thus, in such an embodiment, at least a portion of the bodyside liner 216 may form a part of the gasketing assembly and the gasket element deployable therefrom.

FIGS. 9 and 10 illustrate an absorbent article assembly 240, in accordance with another embodiment of the invention in relaxed and tensioned conditions or states, respectively. More specifically, the absorbent article assembly 240 includes a rear waist section 242 formed, at least in part, with a stretchable material such as in the form of a band 244, a bodyside liner 246, such as described above, with a gasketing assembly 250, in accordance with another embodiment of the invention, disposed therebetween.

The gasketing assembly 250 is generally composed of first and second leg members 252 and 254, respectively. Each of the leg members 252 and 254 includes a first terminal end 256 appropriately joined, connected or secured to the stretchable waist material band 244, such as by means of an adhesive bead 260. Each of the leg members 252 and 254 also includes a second terminal end 262, opposite the first terminal end 256, and appropriately joined, connected or secured to the bodyside liner face 246 such as by means of an adhesive bead 264.

As shown in FIG. 9, in the relaxed condition or state, the leg members 252 and 254 generally lie parallel to and with the stretchable waist material band 244 and the bodyside liner 246. However, upon tensioning of the stretchable waist material 244, the leg members 252 and 254 assume a displacement having at least a component perpendicular to the stretchable waist material 244 and the bodyside liner 246. As a result, at least a portion of the bodyside liner 246, designated by the reference numeral 266, is thrust or otherwise projected in a direction away from the stretchable band 244 and towards, preferably adjacent, the body of the wearer. In practice, such deployment of the bodyside liner portion 266 adjacent the body of the wearer such as to occupy or otherwise fill the volume normally occurring between the waist band of the absorbent article and the small of the back of the wearer serves to reduce the potential for leakage from the absorbent article at such location. Again, in such embodiment, at least a portion of the bodyside liner may form a part of the gasketing assembly and the gasket element deployable therefrom.

While the invention has been described above relative to specific embodiments which generally employ a single gasketing assembly, it is to be understood that the broader practice of the invention is not necessarily so limited. For example, if desired or required such as to permit a more complete or effective minimization or avoidance of leakage, an absorbent article in accordance with the invention may include or incorporate multiple gasketing assemblies.

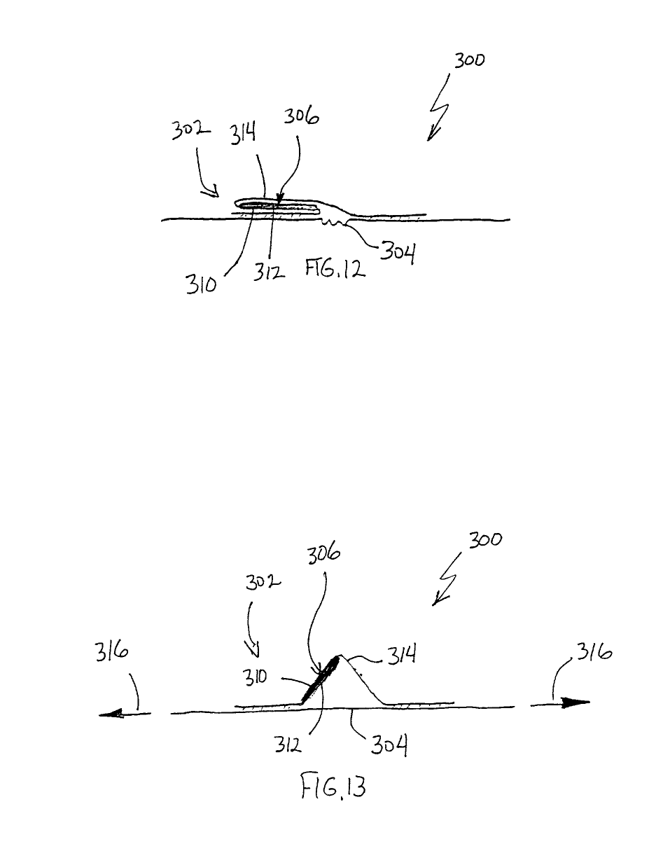

FIG. 11 illustrates one example of such an absorbent article assembly, generally designated by the reference numeral 270.

More specifically, the absorbent article assembly 270 includes a rear waist section 272 formed, at least in part, of a stretchable material 274, such as in the form of a band 276. The absorbent article assembly 270 additionally includes a bodyside liner 280, such as described above, with a plurality of gasketing assemblies 282, such as variously described above, disposed therebetween.

In the absorbent article assembly 270, tensioning of the stretchable band 276 generally results in the gasketing assemblies 282 pushing or deploying the bodyside liner 280 towards, preferably adjacent, the body of the wearer and such as may form or create a containment volume 284, in accordance with the invention.

As will be appreciated, the inclusion of such a plurality of gasketing assemblies linearly displaced along the rear waist portion of an absorbent article can effect a more even or uniform deployment of the bodyside liner towards, preferably adjacent, the body of the wearer, and such as may effectively or completely minimize, avoid or preclude leakage of body exudates between the body, e.g., waist, of the wearer and the waist portion of the absorbent article.

While the invention has been described above in general reference to embodiments containing multiple leg or thrust portions which include or contain a compression resistant member and such as may desirably serve to thrust or deploy an associated portion of the gasketing assembly toward the waist of the wearer, the broader practice of the invention is not necessarily so limited. For example, FIGS. 12 and 13 illustrate an absorbent article assembly 300, in accordance with another embodiment of the invention in relaxed and tensioned conditions or states, respectively. More specifically, the absorbent article assembly 300 is generally similar to the absorbent article assembly 120 described above in that it includes a rear waist section 302 formed, at least in part, of a stretchable material band 304, such as described above, with a gasket assembly member 306 joined thereto. The gasket assembly 306, however, only includes a single leg or thrust portion 310 having a rigid or otherwise compression resistant member 312 and such as may serve to deploy a face portion, designated by the reference numeral 314, toward the waist of the wearer upon tensioning of the stretchable band 304, as signified by the arrows 316 shown in FIG. 13.

It is to be understood that the invention and the principles thereof are, in application, capable of taking various physical forms. Further, it is to be understood absorbent articles in accordance with the invention are generally configured to remove exudates such as runny fecal material from the skin of the wearer and inhibit the longitudinal flow of fecal exudates into regions of the associated absorbent article, such as the rear waist section 32 of the diaper 20 shown in FIG. 1, thus more effectively containing such material within the diaper 20. Still further, it is to be understood that various of the aspects and configurations of the invention can provide distinctive combinations of softness, body conformity, reduced red-marking of the wearer's skin, reduced skin hydration, and improved containment of body exudates.

Thus, the invention provides absorbent articles, particularly in the nature of disposable absorbent articles such as diapers, for example, which in various forms may be more effective in preventing or avoiding the passage of human discharge along the bodyfacing surface of the absorbent article than previously or otherwise obtainable.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. An absorbent article adapted to fit about the waist of a wearer, the absorbent article having a longitudinal direction and a lateral direction, the article comprising:
front and rear waist sections with at least a first portion of the rear waist section formed of a stretchable material,
an intermediate section which includes an absorbent portion and which intermediate section interconnects the front and rear waist sections, and
a gasketing assembly operatively joined with the first portion of the rear waist section to mechanically deploy a gasket element upon tensioning of the stretchable material, the gasket element configured to inhibit a longitudinal flow of human discharge along a bodyfacing surface of the absorbent article.

2. The absorbent article of claim 1 having a longitudinal centerline wherein the gasket element is centered about the longitudinal centerline.

3. The absorbent article of claim 1 wherein the gasket element comprises a face portion deployable toward the waist of the wearer and at least one thrust portion effective to deploy the face portion toward the waist of the wearer.

4. The absorbent article of claim 3 wherein the at least one thrust portion comprises a compression resistant member.

5. The absorbent article of claim 3 wherein the compression resistant member is encased within a soft covering.

6. The absorbent article of claim 3 wherein the gasket element comprises at least a pair of opposed thrust portions effective to deploy the face portion toward the waist of the wearer.

7. The absorbent article of claim 6 wherein each of the pair of opposed thrust portions comprises a compression resistant member.

8. The absorbent article of claim 7 wherein the compression resistant member of each of the pair of opposed thrust portions is encased within a soft covering.

9. The absorbent article of claim 1 wherein a first longitudinal edge of the gasket element is joined at the rear waist section to form a closed rear waist end portion.

10. The absorbent article of claim 9 wherein the gasket element is effective to form a containment volume effective to contain human discharge therewithin.

11. The absorbent article of claim 10 wherein the gasket element is effective to contain human discharge spaced apart from contact with the body of the wearer.

12. The absorbent article of claim 1 wherein the gasketing assembly comprises a plurality of gasket elements.

13. The absorbent article of claim 1 additionally comprising a bodyside liner deployable by action of the gasketing assembly toward the waist of the wearer.

14. The absorbent article of claim 13 wherein the gasketing assembly comprises a pair of leg portions each having a first terminal end connected to a face of the bodyside liner.

15. The absorbent article of claim 13 wherein the gasketing assembly comprises a pair of leg members, each leg member having a first terminal end connected to a face of the bodyside liner and a second terminal end connected to the first portion of the rear waist section formed of a stretchable material.

16. The absorbent article of claim 13 wherein the gasketing assembly comprises a plurality of gasket elements.

17. In a disposable absorbent article which defines a longitudinal direction with a longitudinal centerline and a lateral direction and which absorbent article includes a stretchable waist material adapted to fit about the waist of a wearer, the improvement comprising:
a gasketing assembly operatively joined about the longitudinal centerline with the stretchable waist material to deploy a gasket element against the lower back of a wearer when the waist material is in a stretched conditioned and to form a containment volume, the deployed gasket element being effective to inhibit flow of matter between the waist of the wearer and the waist material of the absorbent article.

18. The disposable absorbent article of claim 17 wherein the gasket element comprises a face portion and at least a pair of opposed thrust portions effective to deploy the face portion toward the waist of the wearer.

19. The disposable absorbent article of claim 18 wherein at least one of the at least a pair of opposed thrust portions comprises a compression resistant member.

20. The disposable absorbent article of claim 17 wherein the gasketing assembly comprises a plurality of gasket elements.

21. The disposable absorbent article of claim 17 additionally comprising a bodyside liner deployable by action of the gasketing assembly toward the waist of the wearer.

22. In an absorbent article having a longitudinal direction and a lateral direction and which absorbent article includes a front waist section, a stretchable rear waist section, and an intermediate section which interconnects the front and rear waist sections and which intermediate section includes an absorbent portion, a method comprising:
tensioning the stretchable rear waist section to deploy a gasket element for inhibiting a longitudinal flow of human discharge along a bodyfacing surface of the absorbent article.

23. The method of claim 22 wherein the absorbent article has a longitudinal centerline and wherein the gasket element is centered about the longitudinal centerline.

24. The method of claim 22 wherein the gasket element includes a face portion and at least one thrust portion wherein upon tensioning the stretchable rear waist section the face portion is deployed toward the waist of the wearer.

25. The method of claim 22 wherein upon deployment of the gasket element, a containment volume effective to contain human discharge therewithin and spaced apart from contact with the body of the wearer is formed.

26. The method of claim 22 wherein the absorbent article also includes a bodyside liner wherein deployment of the gasket element directs the bodyside liner toward the waist of the wearer.

27. The method of claim 22 wherein the tensioning of the stretchable rear waist section effects deployment of a plurality of gasket elements for inhibiting a longitudinal flow of human discharge along a bodyfacing surface of the absorbent article.
